(19) United States
(12) Patent Application Publication
(10) Pub. No.: US 2023/0079396 A1
(43) Pub. Date: Mar. 16, 2023

(54) METHOD FOR TRACKING FITNESS PROGRESS AND TRAINING VOLUME BY MUSCLE GROUP

(71) Applicant: Chester Michael Sokolowski, Tallahassee, FL (US)

(72) Inventor: Chester Michael Sokolowski, Tallahassee, FL (US)

(21) Appl. No.: 17/931,330

(22) Filed: Sep. 12, 2022

Related U.S. Application Data
(60) Provisional application No. 63/261,124, filed on Sep. 13, 2021.

(51) Int. Cl. A63B 24/00; A63B 71/06

(52) U.S. Cl. CPC A63B 24/0062; A63B 24/0075; A63B 71/0622; A63B 2071/065; A63B 2220/807

(57) ABSTRACT

A computer implemented method for consolidating and visually presenting information related to the physique of the user and their muscle training volume; specifically, tracking weight lifted per muscle group.

### CROSS-REFERENCE TO RELATED APPLICATION

This application claims the benefit of priority of U.S. provisional application No. 63/261,124, filed Sep. 13, 2021 the contents of which are herein incorporated by reference.

### BACKGROUND OF THE INVENTION

The present invention relates to fitness monitoring systems and, more particularly, a computer implemented method for consolidating and visually presenting information related to the physique of the user and their muscle training volume; specifically, tracking weight lifted per muscle group.

There are many variables that influence how you look, feel, perform, and progress while weight training over time. Training volume is arguably the most critical variable of performance and physique progression. By “training volume”, the applicant means the total pounds of weight loaded on a muscle and/or muscle group(s) during a single weight training session.

Moreover, muscle groups include the following: abdominals, back, biceps, calves, chest, deltoids (front), deltoids (lateral), deltoids (rear), forearms, glutes, hamstrings, quadriceps, obliques, trapezius, triceps, and the like. The abdominal muscle group includes the rectus abdominis and pyramidalis and the oblique muscle group includes the external obliques, internal obliques, and the transversus abdominis muscles. Similarly, the other muscle groups comprise a plurality of muscles well understood in the art. Upper body, lower body, and whole-body muscle groups will be specifically referred to when intended and reference the various muscle groups above the waist, below the waist, and comprising the entire muscular system, respectively.

Currently, there is not a system to track training volume by muscle group. Furthermore, there is no software that records this tracking data and consolidates it with other fitness information related to the user's physical characteristics, physical performance, nutrition, physical activity, sleep, stress, time management and goals, as well as allows users to upload and compare pictures side by side with their tracking data and fitness information. This is because tracking weight training variables that influence a user's physical appearance, both outside and inside the gym and weight lifted per muscle group, is complicated.

Current solutions do not provide one space where users can upload photos, record the many variables that influence their health, their exercise performance, and their physical appearance, as well as gain feedback on their fitness lifestyle adherence, and see how their body changes over time while comparing their photos with the tracking data and fitness information they entered. Additionally, other software does not give users the ability to select how each exercise they perform targets a specific muscle group, track weight training volume per muscle group over time based on the amount of weight they lift per exercise, and thereby allow meaningful analysis of their weight training volume per muscle group tracking data and associated fitness information along with uploaded photos.

In sum, present systems fail to track weight training volume per muscle group by allowing users to adjust how each exercise loads their muscles and record such tracking data per workout. Furthermore, current systems fail to connect and visually present this tracking data with the many other fitness information, both inside of the gym and outside of the gym, that influence someone's physical appearance. Present systems do not allow users to upload photos and other user-related fitness information such as but not limited to recorded bodyweight, body measurements, nutrition intake, sleep quality, physical activity, stress, time management, goals, training variables, track exercise performance, and the like. Thus, present systems fail to bring that data and information to life in an interactive manner, including the presentation of interactive graphs, charts, calendars, and connection with their photos. Present systems fail to allow users to monitor their comprehensive fitness data, in particular training volume by muscle group, over the weeks, months, and years.

As can be seen, there is a need for a computer implemented method for consolidating and visually presenting information related to the physique of the user and their muscle training volume; specifically, selectively modifying and tracking weight lifted per muscle group.

### SUMMARY OF THE INVENTION

The present invention (colloquially known as “The Science of Sculpture Software”) enables users to record and monitor the weight training volume for the various muscle groups of their body. Furthermore, the software enables users to record data related to their health, exercise performance, and physical appearance, then see their data presented in interactive graphs, charts, and calendars, as well as being presented in relation to the personal photos the user uploads. Through the software application embodied in the present invention, a user is enabled to record and consolidate information in one location related to tracking their physical appearance, fitness progress, and workout performance over time, making future projections more predictable and a better understanding of the feedback regarding how these variables are influencing their physique.

The Science of Sculpture Software application will give users one place to record and consolidate their health and fitness information, upload pictures to compare next to their graphically presented data, modify how exercises load their muscle groups, track training volume per muscle group based on the specific exercises the user performs, and receive feedback to give users insight on their health and fitness lifestyle adherence and quality. The organization, presentation, user-specific customization of how exercises load muscles, and interaction with the data will allow users to simplify how they track their health, fitness progress, training overload, and influential physique-related variables and goals that impact their physical appearance.

Unlike other software that fails to allow users to track training volume by muscle group, edit how various exercises load their muscles and muscle groups, and record pieces of information related to a specific person's workout performance, physique, and/or health, the present invention consolidates the relevant information in one platform with an emphasis on variables that influence their physical appearance. This software enables users to understand how the weight they are lifting in the gym loads their body by adjusting how each exercise loads specific muscles and groups of muscles, logging workouts, and tracking training volume by muscle group. Additionally, users can monitor their progress over time and obtain a complete picture of their health and fitness lifestyle because it allows users to compare their uploaded photos side by side with their training data and fitness information in graphical form as well as receive a grading report and interactive calendar that provides feedback based on the above-mentioned physique-related variables.

In one aspect of the present invention, users can edit how exercises load their muscles, record workouts, and track training volume by muscle group based on the data from the recorded workouts.

In another aspect of the present invention, a system for tracking fitness progress includes the following: a user interface; a processor, and a memory comprising computing device-executable instructions that, when executed by the processor, cause the processor to implement: a tracking data module for generating on the user interface: an exercise regime identifier and one or more muscle group identifiers associated with the identified exercise regime; and a distribution graphical control for each identified muscle group, wherein the distribution graphical control is configured to selectively set a distribution value for each identified muscle group; a workout log module for generating on the user interface for each identified exercise regime: one or more weight parameters; and a parameter graphical control element configured to selectively set a weight value for each weight parameter; and the tracking data module configured to represent on the user interface a volume value for each identified muscle group, wherein the volume value is defined as a function of a product of the repetition distribution value and the set weight parameters.

In another aspect of the present invention the system for tracking fitness progress further includes wherein the parameter graphical control element is configured to selectively adjust a set value for each weight parameter, wherein the parameter graphical control element is configured to selectively adjust a repetition value for each weight parameter, wherein each distribution value is initially populated with an initial distribution value, wherein the tracking data module is configured to retrievable store each volume value so that an average volume value is represented on the user interface for an identified average duration of time, wherein the tracking data module is configured to retrievable store each volume value so that a total volume value is represented on the user interface for an identified total duration of time, wherein the computing device-executable instructions cause the processor to implement a comparator module for adding a captured image for an associated duration of time, wherein the tracking data module is configured to represent the captured image juxtaposed to the average volume value and/or the total volume value when the identified total duration of time and the associated duration of time overlap in time, wherein the tracking data module is configured to selectively add and delete a plurality of muscle group identifiers associated with each exercise regime identifier, wherein the one or more muscle groups comprises an abdominal muscle group, wherein the abdominal muscle group comprises the rectus abdominis and pyramidalis muscles.

In still yet another aspect of the present invention, users can view training volume by muscle group next to their progress pictures juxtaposed with other recorded data related to their fitness lifestyle, and gain feedback on their overall fitness progress via the systemic interactive calendar and grading report card.

These and other features, aspects and advantages of the present invention will become better understood with reference to the following drawings, description, and claims.

### BRIEF DESCRIPTION OF THE DRAWINGS

FIG. 1A is a schematic view of an exemplary embodiment of the present invention, illustrating a default setting that is provided by the present invention for determining a weight loading distribution per muscle group per a given weightlifting exercise.

FIG. 1B is a schematic view of an exemplary embodiment of the present invention, illustrating how the user can selectively adjust each weight loading distribution per muscle group for the given weightlifting exercise.

FIG. 1C is a schematic view of an exemplary embodiment of the present invention, illustrating how the user can further selectively adjust the tracking data of the weight loading distribution associated with the given weightlifting exercise by editing the muscle groups affected.

FIG. 1D is a schematic view of an exemplary embodiment of the present invention, illustrating how the software saves and stores the user-selected distribution of the weight load for each user-identified muscle group for the given weightlifting exercise.

FIG. 2A is a schematic view of an exemplary embodiment of the present invention, illustrating how a given weightlifting exercise loads the user-identified muscle groups.

FIG. 2B is a schematic view of an exemplary embodiment of the present invention, illustrating the user can adjust how each exercise's distribution of the weight lifted to the user-identified muscle groups involved in that exercise, which the software retrievable stores under user preferences.

FIG. 2C is a schematic view of an exemplary embodiment of the present invention, illustrating a user interface for a user to log the tracking data into a workout log module for determining a “volume” of distributed weight for one or more muscle group involved in the given exercise over a user-selected duration (e.g., week, month, year, etc.) of that given exercise.

FIG. 2D is a schematic view of an exemplary embodiment of the present invention, illustrating the tracking data functionality for training volume by muscle group.

FIG. 3 is a schematic view of an exemplary embodiment of the present invention, illustrating a comparator functionality for data (e.g., training volume for the chest muscle group) next to uploaded images (progress pictures). Note, “AVG” may be the average per workout, while the “TOTAL” refers to a summation over a user-specified duration; frequently they will have different values.

FIG. 4 is a schematic view of an exemplary embodiment of the present invention, illustrating the comparator functionality for fitness information (e.g., bodyweight) adjacent to and uploaded images (progress pictures) for a specific time/day.

FIG. 5 is a schematic view of an exemplary embodiment of the present invention, illustrating an interactive calendar functionality and grading scale functionality adapted to give rapid feedback on a specific goal through color coding (e.g., a grade of A/B results in a red color with the circles associated with a specific day represented on the interactive calendar functionality and grading scale functionality).

FIG. 6 is a flow chart for an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a computer implemented method for consolidating and visually presenting information related to the physique of the user and their muscle training volume; specifically, tracking weight lifted per muscle group.

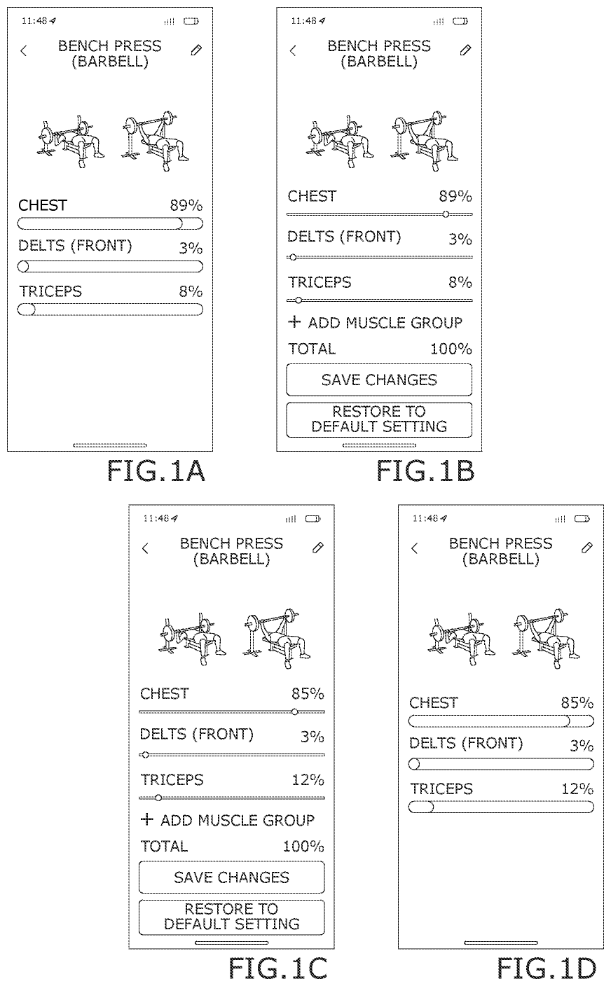

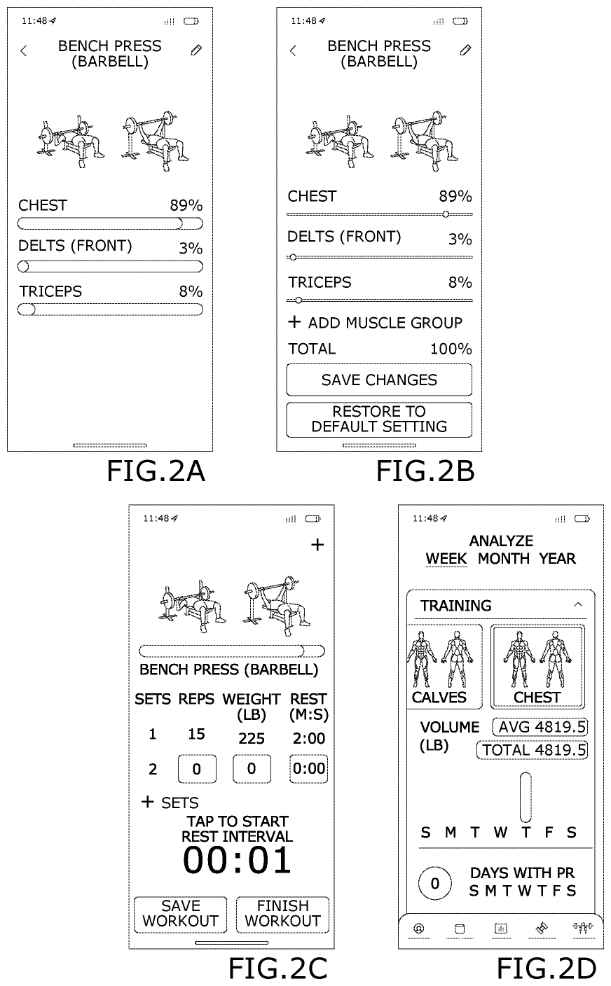

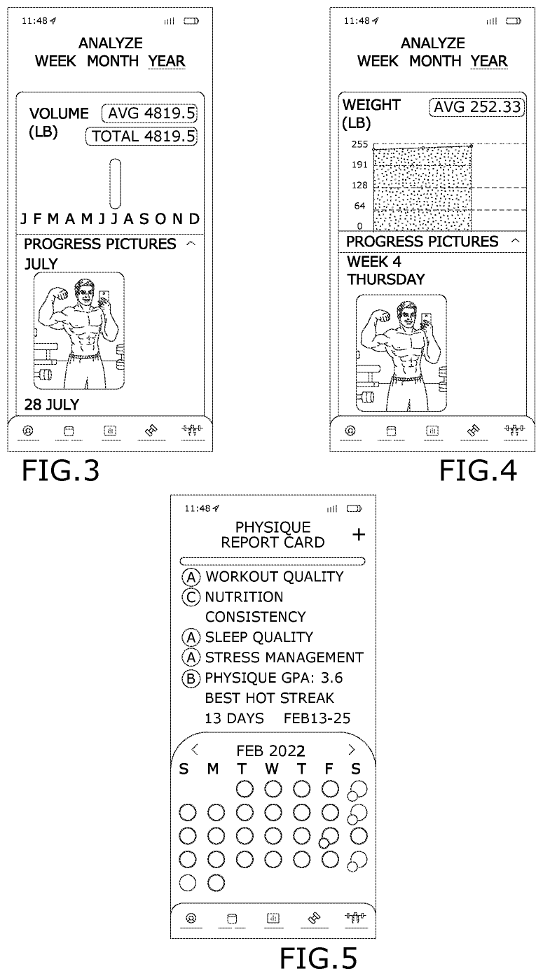

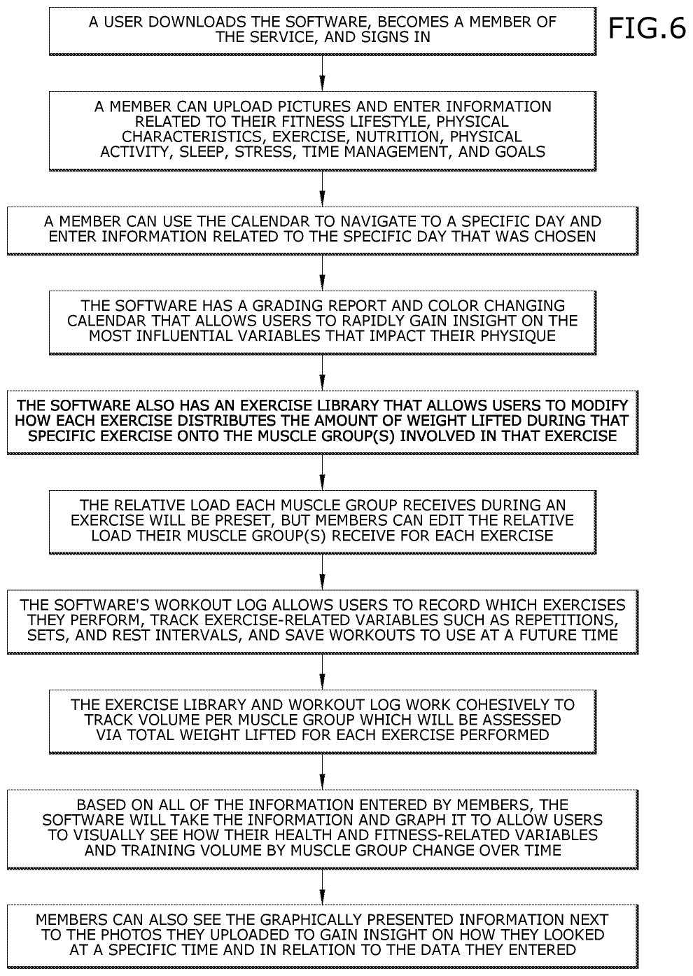

Referring now to FIGS. 1A through 6, the present invention may include one or more servers and at least one computer. Each server and computer of the present invention may each include computing systems. This disclosure contemplates any suitable number of computing systems. This disclosure contemplates the computing system taking any suitable physical form. As example and not by way of limitation, the computing system may be a virtual machine (VM), an embedded computing system, a system-on-chip (SOC), a single-board computing system (SBC) (e.g., a computer-on-module (COM) or system-on-module (SOM)), a desktop computing system, a laptop or notebook computing system, a smart phone, an interactive kiosk, a mainframe, a mesh of computing systems, a server, an application server, or a combination of two or more of these. Where appropriate, the computing systems may include one or more computing systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

In certain embodiments, the network may refer to any interconnecting system capable of transmitting audio, video, signals, data, messages, or any combination of the preceding. The network may include all or a portion of a public switched telephone network (PSTN), a public or private data network, a local area network (LAN), a metropolitan area network (MAN), a wide area network (WAN), a local, regional, or global communication or computer network such as the Internet, a wireline or wireless network, an enterprise intranet, or any other suitable communication link, including combinations thereof.

In some embodiments, the computing systems may execute any suitable operating system such as IBM's zSeries/Operating System (z/OS), MS-DOS, PC-DOS, MAC-OS, WINDOWS, UNIX, OpenVMS, an operating system based on LINUX, or any other appropriate operating system, including future operating systems. In some embodiments, the computing systems may be a web server running web server applications such as Apache, Microsoft's Internet Information Server™, and the like.

In particular embodiments, the computing systems includes a processor, a memory, a user interface and a communication interface. In particular embodiments, the processor includes hardware for executing instructions, such as those making up a computer program. The memory includes main memory for storing instructions such as computer program(s) for the processor to execute, or data for processor to operate on. The memory may include mass storage for data and instructions such as the computer program. As an example, and not by way of limitation, the memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to computing system, where appropriate. In particular embodiments, the memory is non-volatile, solid-state memory.

The user interface includes hardware, software, or both providing one or more interfaces for communication between a person and the computer systems. As an example, and not by way of limitation, a user interface device may include a keyboard, keypad, microphone, monitor, mouse, printer, scanner, speaker, still camera, stylus, tablet, touchscreen, trackball, video camera, another suitable user interface or a combination of two or more of these. A user interface may include one or more sensors. This disclosure contemplates any suitable user interface and any suitable user interfaces for them.

The communication interface includes hardware, software, or both providing one or more interfaces for communication (e.g., packet-based communication) between the computing systems over the network. As an example, and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface. As an example and not by way of limitation, the computing systems may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing systems may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing systems may include any suitable communication interface for any of these networks, where appropriate.

Software users will download and access the software on their computer (e.g., mobile phone). Software users enter their tracking data and modify how exercises load their muscle groups. Users will record their health and fitness information on a regular basis, including upload photos of themselves to display health and fitness progress over time, record parameters related to their workouts to form a workout log and may modify how each exercise regimen distributes load to one or their muscle groups. Pictures of the exercises may be displayed which will help guide the user when building workouts to form the workout log and adjusting how each exercise loads their muscles.

The user interface is configured for projecting different colors and high-resolution images. For instance, a graded report card functionality and interactive calendar functionality may represent grades for the user's tracking data and fitness information through a rubric wherein red is equated with good behavior to promote fast physique progress, wherein purple is equated with neutral behavior to promote slow physique progress, and wherein blue is equated with poor behavior to negate physique progress Based on the information inputted into the software, colorful graphs, charts, and a calendar would display and otherwise visually present the data as it changes over time. The software application user can also examine their uploaded photos side by side with their graphs and charts to monitor how their body changes over the weeks, months, and years.

Overall, the system may include users becoming systemic members to use the systemic software application disclosed herein. Once a member, users can access the software through their mobile device then edit the distribution of load on one or more muscle groups for one or more exercises or workouts. Furthermore, users can upload health and fitness information about themselves, including information related their fitness lifestyle, physical characteristics, exercise, nutrition, physical activity, sleep, stress, time management, and goals. Members can upload pictures via their computing devices image capturing or light sensing device, for instance from their mobile device's image roll.

Members will use the interactive calendar to navigate to a specific day and voluntarily enter fitness information related to the specific day that was chosen.

To access the exercise library and workout log, users will navigate to the specific tab. The exercise library allows users to modify how each exercise distributes the amount of weight lifted during that specific exercise onto one or more muscle groups. The user can select from a list directly provided by the software. The relative load (of the total weight lifted during the relevant workout) that each muscle group receives during an exercise will be preset, but members can edit the relative load a selected muscle group(s) receive for each exercise through use of a slider or track bar or other graphical control element with which a user may set a value by selectively changing an indicator or other value identified.

The workout log allows users to record which exercises they perform, thereby tracking exercise-related variables or tracking data parameters such as repetitions, sets, and rest intervals, and save workouts to use at a future time. The exercise library and workout log work cohesively to track training volume per muscle group which will be assessed via weight lifted each set for each exercise performed (e.g., volume=weight lifted set 1*reps set 1+weight lifted set 2*reps set 2, etc.). Volume may equal the weight lifted (measured in pounds or kilograms), wherein training volume may equal the total amount of weight a user lifted during an identified workout. The present invention tracks the total amount of weight a user lifts and the distribution of that total amount of weight across one or more muscle groups.

Based on all the information entered by members, the software will take the tracking data and graph it to allow users to see how their health and fitness-related variables and training volume by muscle group change over time. Members can also see the graphically presented information next to the photos they uploaded, thereby gaining insight on how they looked at a specific time and in relation to the tracking data and fitness information they entered. In addition, the software has a grading report and color changing calendar that allows users to rapidly gain insight on the most influential variables that impact their physique (workout quality, nutrition consistency, sleep quantity, and stress management).

The present invention revolutionizes the way users can quickly view the wide scope of consolidated, relevant information related to altering their physique side by side with the photos of their physique, thereby simplifying how users monitor their health, fitness lifestyle, workout related variables, and progress over months, weeks, and years. Most importantly, the software allows users to edit how exercises load their muscles based on their perceived preference, then track how the weight they lift is distributed about their working muscles.

Ideally, the user consistently enters in their personal data into the software, uploads pictures, and records workouts in order to see graphs of their data, receives feedback on the interactive calendar of their general fitness lifestyle commitment, and monitors their progress over time. The feedback includes interpretation of their data; for example, based on their data, system gives users color coded feedback (red, purple, blue) and letter grades (A, B, C, D, F) on the calendar and grading report, respectively, for their behaviors based on whether the behaviors promote positive and negative physique changes.

In addition, the person must manually adjust how the exercises load their specific muscle group(s) if the person wishes to change how each muscle is loaded and deviate from the preset recordings of relative load distributed to the muscle groups involved in each exercise. In summation, if the user enters a limited amount of data into the software application, a limited number of user-specific results, feedback, and interaction will display.

To make the present invention, one must build software that is able to complete the requisite tasks and provide the user with the useful tools described hereabove. The primary features that track training volume by muscle group then consolidate health and fitness information in one location are the: 1) computing device to access the software, 2) exercise library containing the picture of the exercise and relative load each exercise places on the muscle group(s) involved in the exercise that can be customized by each specific member, 3) workout log for members to record, build, and save workout related information, 4) interactive calendar where users navigate to a specific date to upload photos, record information related to their physical characteristics, exercise, nutrition, physical activity, sleep, stress, and time management, and receive feedback based on their adherence to and quality of their training, nutrition, sleep, and stress, and 5) graphs and charts containing the information voluntarily submitted by members.

To make the present invention work better, higher screen resolution and more vivid color screen resolution will help the user perceive the visual information that is presented in the interactive calendar, graphs, charts, and their personal uploaded photos. The software can also be made better by synchronizing it with other software that record individual elements of health and fitness lifestyle variables such as smart watches that specifically capture sleep or other software-loaded devices that records step count, etc.

The present invention is a tool where users can record and track physique-influencing information and variables via graphs and charts in relation and juxtaposed to associated photos of said physique. Some embodiments could swap what specific information is recorded and graphed to compare next user uploaded photos. For example, this software records and graphs nutrition information relating to caloric intake, macronutrients, supplements, alcohol, snacks, and meal adherence, and another software could ask for different variables related to a user's sport performance, dietary intakes, supplements, and/or drugs consumed. This software may also record training volume by muscle group, while another software could redefine the muscle groups or regions of the body where training load is distributed to. For example, instead of tracking data on abdominals and obliques, the present invention could label the muscle groups in another manner (e.g., core) and track the volume of said muscle group. Altering what specific fitness information is recorded and how muscle volume is defined and tracked are ways to reconfigure the information obtained that will be consolidated to one area like this software does.

In addition, the concept of a calendar and grading scale based on only the most pertinent, influential variables to determine a specific outcome or related to achieving a specific goal can be copied and adjusted to fit another desired outcome or goal. The desired outcome could be related to disease risk, disease progression, and/or disease treatment, or any other desired health and fitness-related outcome. For example, instead of making a grading scale based on workout quality, nutrition, sleep, and stress to gauge a successful physical appearance transformation, another scale composed of blood sugar control, exercise, meal timing, waist circumference, and medication dose could be used to gauge diabetes management and progression. The use of an interactive calendar and report card to give feedback on a desired outcome and goal can be molded to fit many different outcomes and goals based on the specific criteria assessed.

A method of using the present invention may include the following. Over the course of many weeks, months, and years, the user would consistently enter in their data relating to their “physique variables” including physical characteristics, exercise, nutrition, physical activity, sleep, stress, time management, and goals as well as upload photos of their physique to monitor how it is changing over time as a function of the inputted “physique variables”. Additionally, the user would adjust the relative load each exercise places on their muscle group(s) based upon their subjective feelings and individual technique, consistently record their workouts, and track their training performance over time. By consistently inputting their data into The Science of Sculpture Software, the user is enabled to build a library of relevant information relating to changing their health, performance, and physique. When data is entered into the software on a consistent basis and over the course of many weeks, months, and years, the software will translate that information to graphs, charts, the color changing calendar, and the physique report card. The user can view the graphs, charts, calendar, and report card to gain knowledge on what is influencing their health, performance, and physique over a selected portion of or entire period they have been entering data into the software. The more data that is entered by the user and the longer the user records data into the software, the more feedback they will receive, and thus they'll have more insight on what variables alter their health, fitness progress, and physique. The user can learn what influences their overall health and fitness progress, spot trends, and make more knowledgeable decisions when choosing which changes they want to make to their lifestyle and physical training in order to improve their health, expedite their physique progress, and sustain the beneficial results.

The problem of tracking the many variables that influence a user's health and fitness progress, especially training volume by muscle group and physical aesthetic progress, is made easier by The Science of Sculpture Software because it captures a wide range of relevant data both inside and outside of the gym, allows users to upload photos to go along with their data, and provides visual feedback in the form of graphs, charts, a color responsive calendar, and a graded report card containing the most pertinent physique influencing variables. Formulas programmed in the software that respond to user's data can result in self-grading. For example, nutrition if someone plans 6 meals and eats 5, they hit ⅚ meals which is 83% which translates to a B on nutrition consistency.

As used in this application, the term “about” or “approximately” refers to a range of values within plus or minus 10% of the specified number. And the term “substantially” refers to up to 80% or more of an entirety.

It should be understood, of course, that the foregoing relates to exemplary embodiments of the invention and that modifications may be made without departing from the spirit and scope of the invention as set forth in the following claims.

What is claimed is:

1. A system for tracking fitness progress, the system comprising:
a user interface;
a processor, and
a memory comprising computing device-executable instructions that, when executed by the processor, cause the processor to implement:
a tracking data module for generating on the user interface:
an exercise regime identifier and one or more muscle group identifiers associated with the identified exercise regime; and
a distribution graphical control for each identified muscle group, wherein the distribution graphical control is configured to selectively set a distribution value for each identified muscle group;
a workout log module for generating on the user interface for each identified exercise regime:
one or more weight parameters; and
a parameter graphical control element configured to selectively set a weight value for each weight parameter; and
the tracking data module configured to represent on the user interface a volume value for each identified muscle group, wherein the volume value is defined as a function of a product of the repetition distribution value and the set weight parameters.

2. The system of claim 1, wherein the parameter graphical control element is configured to selectively adjust a set value for each weight parameter.

3. The system of claim 2, wherein the parameter graphical control element is configured to selectively adjust a repetition value for each weight parameter.

4. The system of claim 3, wherein each distribution value is initially populated with an initial distribution value.

5. The system of claim 4, wherein the tracking data module is configured to retrievable store each volume value so that an average volume value is represented on the user interface for an identified average duration of time.

6. The system of claim 5, wherein the tracking data module is configured to retrievable store each volume value so that a total volume value is represented on the user interface for an identified total duration of time.

7. The system of claim 6, wherein the computing device-executable instructions cause the processor to implement a comparator module for adding a captured image for an associated duration of time.

8. The system of claim 7, wherein the tracking data module is configured to represent the captured image juxtaposed to the average volume value and/or the total volume value when the identified total duration of time and the associated duration of time overlap in time.

9. The system of claim 8, wherein the tracking data module is configured to selectively add and delete a plurality of muscle group identifiers associated with each exercise regime identifier.

10. The system of claim 9, wherein the one or more muscle groups comprises an abdominal muscle group.

11. The system of claim 10, wherein the abdominal muscle group comprises rectus abdominis and pyramidalis muscles.
